(19) United States
(12) Patent Application Publication
(10) Pub. No.: US 2024/0404668 A1
(43) Pub. Date: Dec. 5, 2024

(54) MEDICAL INFORMATION PROCESSING DEVICE, MEDICAL INFORMATION PROCESSING SYSTEM, AND MEDICAL INFORMATION PROCESSING METHOD

(71) Applicant: CANON MEDICAL SYSTEMS CORPORATION, Otawara-shi (JP)

(72) Inventors: Taro TAKOSHIMA, Nasushiobara (JP); Ichirou MAEDA, Otawara (JP); Koichi TERAI, Shioya (JP); Masahiro YOSHIWAKI, Otawara (JP); Hiroyuki YAMASAKI, Yaita (JP); Masato SHIBUYA, Otawara (JP)

(73) Assignee: CANON MEDICAL SYSTEMS CORPORATION, Otawara-shi (JP)

(21) Appl. No.: 18/676,641

(22) Filed: May 29, 2024

(30) Foreign Application Priority Data
May 30, 2023 (JP) 2023-088611

(51) Int. Cl. G16H 15/00; G16H 10/20

(52) U.S. Cl. CPC G16H 15/00; G16H 10/20

(57) ABSTRACT

A medical information processing device of an embodiment includes processing circuitry. The processing circuitry is configured to acquire clinical data of a subject, and select one or more analysis applications that analyze the acquired clinical data on the basis of report data of a user regarding analysis results of a plurality of types of analysis applications that analyze clinical data.

### CROSS-REFERENCE TO RELATED APPLICATION

The present application claims priority based on Japanese Patent Application No. 2023-088611 filed May 30, 2023, the content of which is incorporated herein by reference.

### FIELD

Embodiments disclosed in this specification and drawings relate to a medical information processing device, a medical information processing system, and a medical information processing method.

### BACKGROUND

Analysis applications that analyze clinical data such as medical images captured by a medical image diagnostic device and obtain desired analysis results (hereinafter referred to as “analysis applications”) have been known. By referring to analysis results obtained from analysis applications, doctors can diagnose patients and create reports of diagnosis results accurately and smoothly.

There are various types of analysis applications depending on the types of clinical data to be analyzed and purposes of analysis. It is desirable to preferentially select an analysis application suitable for diagnosis and provide useful analysis results to doctors. However, in order to automatically select an analysis application, a mechanism of receiving feedback from a doctor regarding the usefulness of analysis results and selecting an appropriate application from among a plurality of analysis applications on the basis of the feedback is needed.

### BRIEF DESCRIPTION OF THE DRAWINGS

FIG. 1 is a diagram showing an example of a configuration of a medical information processing system S according to an embodiment.

FIG. 2 is a functional block diagram showing an example of a configuration of an analysis server 1 according to the embodiment.

FIG. 3A is a diagram showing an example of input and output data of a learning model MD1 according to the embodiment.

FIG. 3B is a diagram showing an example of input and output data of a learning model MD2 according to the embodiment.

FIG. 3C is a diagram showing an example of input and output data of a learning model MD3 according to the embodiment.

FIG. 4 is a functional block diagram showing an example of a configuration of a terminal device 3 according to the embodiment.

FIG. 5 is a diagram showing an example of report data RD stored in a report system 7 according to the embodiment.

FIG. 6 is a sequence diagram showing an example of processing of registering report data RD in the medical information processing system S according to the embodiment.

FIG. 7 is a diagram showing an example of a report creation screen P1 displayed on the terminal device 3 according to the embodiment.

FIG. 8 is a sequence diagram showing an example of learning processing in the medical information processing system S according to the embodiment.

### DETAILED DESCRIPTION

Hereinafter, a medical information processing device, a medical information processing system, and a medical information processing method according to an embodiment will be described with reference to the drawings. The medical information processing system of an embodiment acquires feedback from users regarding the accuracy and usefulness of analysis results of various analysis applications and makes it possible to select an optimal analysis application depending on the situation on the basis of the feedback.

A medical information processing device of an embodiment includes processing circuitry. The processing circuitry is configured to acquire clinical data of a subject, and select one or more analysis applications that analyze the acquired clinical data on the basis of user report data on analysis results of a plurality of types of analysis applications that analyze clinical data.

### [Configuration of Medical Information Processing System]

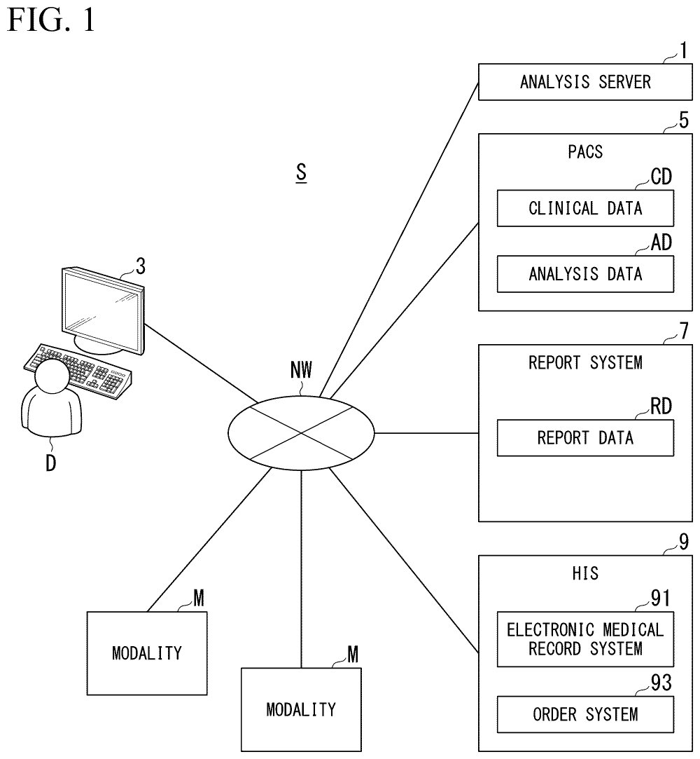

FIG. 1 is a diagram showing an example of a configuration of a medical information processing system S according to an embodiment. The medical information processing system S includes, for example, an analysis server 1, a terminal device 3, a picture archiving and communication system (PACS) 5, a report system 7, a hospital information system (HIS) 9, and one or more modalities M. The analysis server 1, the terminal device 3, the PACS 5, the report system 7, the HIS 9, and the modalities M are connected to each other via a communication network NW such that they can communicate. The communication network NW is any information communication network using telecommunications technology. For example, the communication network NW includes a wireless/wired LAN such as a hospital backbone local area network (LAN), an Internet network, a telephone communication line network, an optical fiber communication network, a cable communication network, a satellite communication network, and the like.

### [Modality]

The modality M is a medical device that acquires clinical data of a subject. The modality M includes, for example, an X-ray computed tomography (CT) device, an X-ray diagnostic device, a magnetic resonance imaging device, an ultrasonic diagnostic device, a nuclear medicine diagnostic device, an electrocardiograma pulse meter, and the like. The modality M is operated by, for example, a doctor, a technician, or the like. Clinical data generated by the modality M is transmitted to the analysis server 1 and the PACS 5. An example of a case in which the modality M is an X-ray CT device will be described below.

### [Analysis Server]

The analysis server 1 selects and executes an analysis application on the basis of the clinical data transmitted from the modality M and order information and electronic medical record information transmitted from the HIS 9, and transmits analysis results of the analysis application to the terminal device 3. Furthermore, the analysis server 1 performs processing for selecting an analysis application on the basis of feedback information transmitted from the report system 7. The analysis server 1 is an example of a “medical information processing device.”

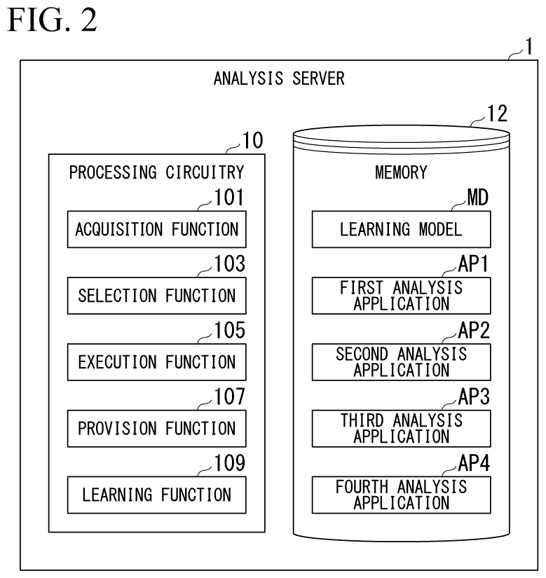

FIG. 2 is a functional block diagram showing an example of a configuration of the analysis server 1 according to the embodiment. The analysis server 1 includes, for example, processing circuitry 10 and a memory 12. The processing circuitry 10 controls the overall operation of the analysis server 1. The processing circuitry 10 executes, for example, an acquisition function 101, a selection function 103, an execution function 105, a provision function 107, and a learning function 109. These functions are realized, for example, by a hardware processor (computer) executing a program (software) stored in the memory 12. The hardware processor is, for example, circuitry such as a central processing unit (CPU), a graphics processing unit (GPU), an application specific integrated circuit (ASIC), or a programmable logic device (for example, a simple programmable logic device (SPLD), a complex programmable logic device (CPLDs), or a field programmable gate array (FPGA)). The program may be directly incorporated into the circuit of the hardware processor instead of being stored in the memory 12. In this case, the hardware processor realizes the functions by reading and executing the program incorporated into the circuit. The hardware processor is not limited to being configured as a single circuit, but may be configured as one hardware processor by combining a plurality of independent circuits to realize each function. Further, a plurality of components may be integrated into one hardware processor to realize each function.

The acquisition function 101 acquires clinical data transmitted from the modality M and order information and electronic medical record information transmitted from the HIS 9. The acquisition function 101 also acquires feedback information (user report data) transmitted from the report system 7. The acquisition function 101 is an example of an “acquirer.”

The selection function 103 selects one or more analysis applications that analyze the clinical data acquired by the acquisition function 101 from among a plurality of types of analysis applications stored in advance in the memory 12 on the basis of feedback information regarding analysis results of the plurality of types of analysis applications that analyze clinical data (user report data, the type of an analysis application that has generated analysis results selected by a user, and the type of an analysis application that has generated analysis results not selected by the user). For example, when the clinical data is CT image data, the selection function 103 selects an analysis application according to information in a format compliant with the Digital Imaging and Communication in Medicine (DICOM) standard (hereinafter also referred to as a “DICOM tag”) attached to the CT image data. The DICOM tag includes information such as modality information, imaging conditions (target region information of an examination target, an imaging protocol, and the like), an examination ID, and a subject ID. The selection function 103 is an example of a “selector.”

Further, the selection function 103 selects one or more analysis applications on the basis of supplementary information associated with analysis results included in report data in addition to the types of analysis applications. The supplementary information includes at least one of information indicating diagnosis (a suspected disease name and the like) of a user and order information of clinical data.

The selection function 103 selects an analysis application using, for example, a learning model MD stored in advance in the memory 12. The learning model MD is a model that has been trained to output candidates for analysis applications to be executed (hereinafter referred to as “analysis application candidates”) when clinical data, order information, electronic medical record information, and the like are input. The learning model MD is generated using various description methods such as a neural network, a support vector machine, and a decision tree. Neural networks include, for example, an auto-encoder, a convolutional neural network (CNN), a recurrent neural network (RNN), and the like. The selection function 103 inputs clinical data, order information, electronic medical record information, and the like to the learning model MD and obtains analysis application candidates output from the learning model MD.

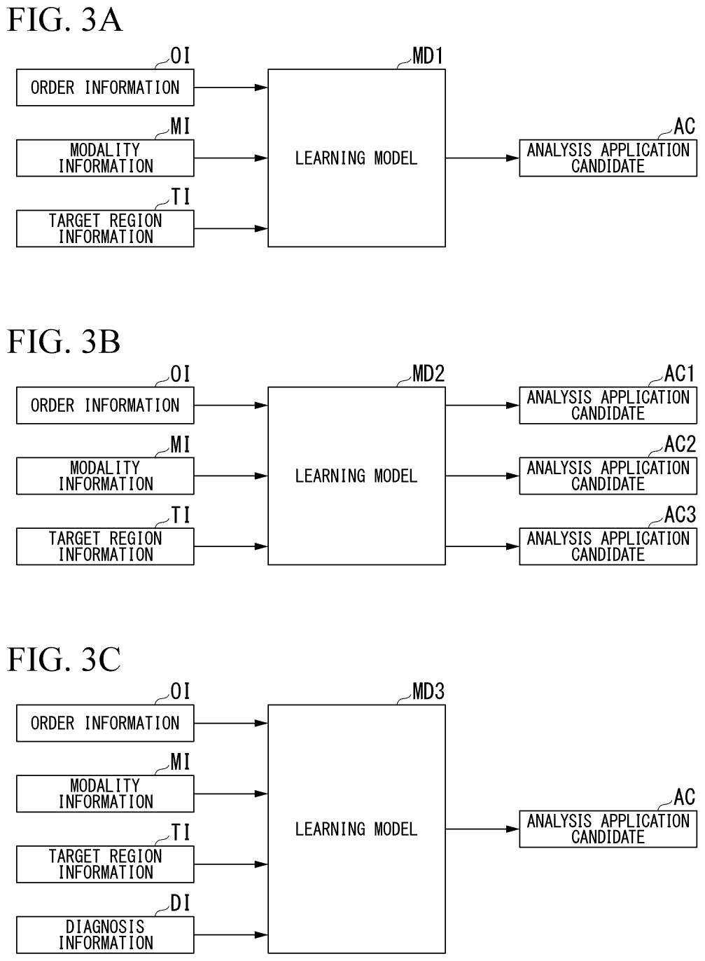

FIG. 3A is a diagram showing an example of input and output data of a learning model MD1 according to an embodiment. The learning model MD1 has order information OI, modality information MI, and target region information TI as input data, and has one analysis application candidate AC as output data. The order information OI includes, for example, an examination purpose. The modality information MI is information for identifying a modality (image type) acquired from a DICOM tag. The target region information TI is information indicating a region to be examined, which is acquired from the DICOM tag.

FIG. 3B is a diagram showing an example of input and output data of a learning model MD2 according to an embodiment. The learning model MD2 has order information OI, modality information MI, and target region information TI as input data, and has three analysis application candidates AC1 to AC3 as output data. The three analysis application candidates AC1 to AC3 may be prioritized using a neural network softmax function or the like. The number of analysis application candidates to be output data is arbitrary, and may be two, four or more.

FIG. 3C is a diagram showing an example of input and output data of a learning model MD3 according to an embodiment. The learning model MD3 has diagnosis information DI as input data in addition to order information OI, modality information MI, and target region information TI, and has one analysis application candidate AC as output data. The diagnosis information DI is, for example, information indicating a doctor's diagnosis result regarding a medical condition of a subject obtained from order information or electronic medical record information. The number of analysis application candidates to be output data is arbitrary, and may be two or more.

That is, the selection function 103 selects, as one or more analysis applications, candidates for analysis applications output from the learning model MD by inputting acquired clinical data to the learning model MD trained to output candidates for analysis applications that analyze clinical data when the clinical data is input.

The selection function 103 may select one or more analysis applications on the basis of statistical data of an analysis application that has generated analysis results included in report data (statistical data of an analysis application that was an output source of an analysis result selected as a key image which will be described later) or statistical data of an analysis application that has generated analysis results that are not included in the report data (statistical data of an analysis application that was an output source of an analysis result that was not selected as the key image which will be described later) instead of a learning model based on machine learning technology.

Referring back to FIG. 2, the execution function 105 executes an analysis application selected by the selection function 103. When a plurality of analysis applications are selected by the selection function 103, the execution function 105 executes each of the plurality of analysis applications. The execution function 105 is an example of an “executor.”

The provision function 107 transmits analysis results output from the analysis application executed by the execution function 105 to the terminal device 3. As a result, the analysis results are displayed on the terminal device 3. Further, the provision function 107 may transmit clinical data (CT image data) acquired by the acquisition function 101 to the terminal device 3. The provision function 107 is an example of a “provider.”

The learning function 109 generates a learning model MD by performing learning processing using feedback information (report data) from the report system 7 acquired by the acquisition function 101 and stores the learning model MD in the memory 12. The learning function 109 is an example of a “learner.” Details of processing of the learning function 109 will be described later.

The memory 12 is realized by, for example, a semiconductor memory element such as a random access memory (RAM) or a flash memory, a hard disk, an optical disc, or the like. The memory 12 stores, for example, the learning model MD, a first analysis application AP1, a second analysis application AP2, a third analysis application AP3, a fourth analysis application AP4, and the like. Such data may be stored in an external memory with which the analysis server 1 can communicate instead of the memory 12 (or in addition to the memory 12). The external memory is controlled by a cloud server, for example, when the cloud server that manages the external memory receives read/write requests.

### [Terminal Device]

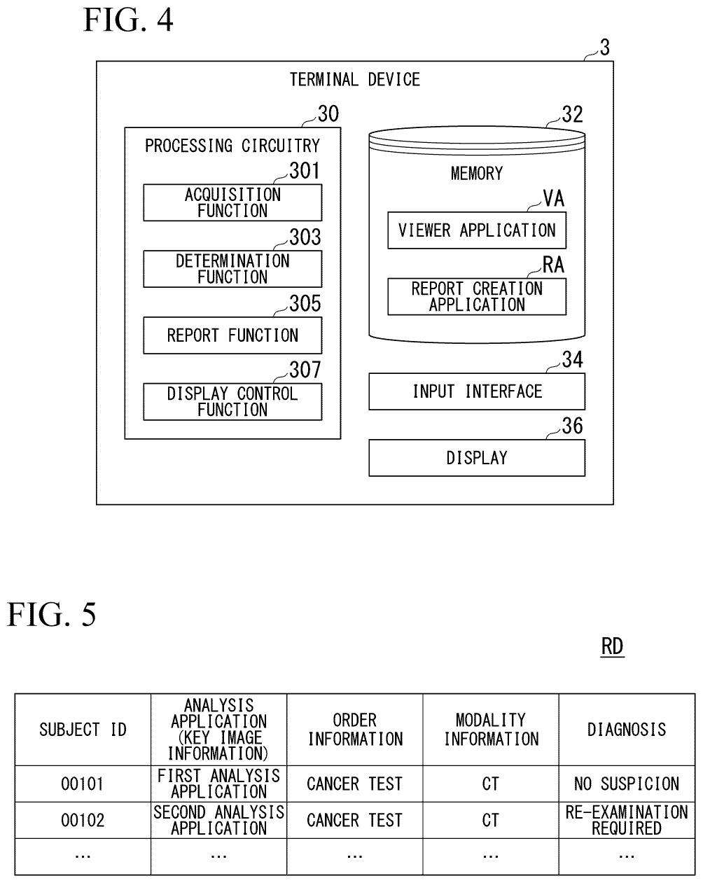

Referring back to FIG. 1, the terminal device 3 is a device for creating report data based on analysis results with reference to the analysis results provided by the analysis server 1. The terminal device 3 is operated by, for example, a user such as a doctor or a technician (hereinafter referred to as a “doctor D”). The terminal device 3 is, for example, a personal computer, a mobile terminal such as a tablet or a smartphone, or the like. FIG. 4 is a functional block diagram showing an example of a configuration of the terminal device 3 according to the embodiment. The terminal device 3 includes, for example, processing circuitry 30, a memory 32, an input interface 34, and a display 36.

The processing circuitry 30 executes, for example, an acquisition function 301, a determination function 303, a report function 305, and a display control function 307. These functions are realized, for example, by a hardware processor (computer) executing a program (software) stored in the memory 32.

The acquisition function 301 acquires analysis results and clinical data (CT image data) transmitted from the analysis server 1.

The determination function 303 determines, as a key image, analysis results (an image) selected by the doctor D as being useful for diagnosis from among analysis results on the basis of an operation of the doctor D via the input interface 34. Details of processing of the determination function 303 will be described later.

The report function 305 creates report data on the basis of an operation of the doctor D via the input interface 34. The report function 305 executes, for example, a report creation application RA stored in advance in the memory 32. The report data includes, for example, a key image determined by the determination function 303, and diagnosis information of the doctor D associated with the key image. Details of processing of the report function 305 will be described later.

The display control function 307 causes the display 36 to display analysis results acquired by the acquisition function 301, graphical user interface (GUI) images for receiving various operations by the doctor D, and the like. For example, the display control function 307 executes a viewer application VA stored in advance in the memory 32 and causes the display 36 to display analysis results.

The memory 32 is realized by, for example, a semiconductor memory element such as a RAM or a flash memory, a hard disk, an optical disc, or the like. The memory 32 stores, for example, the viewer application VA, the report creation application RA, and the like. Such data may be stored in an external memory with which the terminal device 3 can communicate instead of the memory 32 (or in addition to the memory 32).

The input interface 34 receives various input operations from the doctor D, and outputs electrical signals indicating the content of the received input operations to the processing circuitry 30. For example, the input interface 34 receives operations such as selecting a key image and inputting diagnosis information. For example, the input interface 34 is realized by a mouse, a keyboard, a touch panel, a track ball, a switch, a button, a joystick, a camera, an infrared sensor, a microphone, or the like. In this specification, the input interface is not limited to one that includes physical operation parts such as a mouse and a keyboard. For example, examples of the input interface include electrical signal processing circuitry that receives an electrical signal corresponding to an input operation from an external input apparatus provided separately from the device and outputs this electrical signal to a control circuit.

The display 36 displays various types of information. For example, the display 36 displays a view screen generated by the processing circuitry 30, a report creation screen, a GUI image for receiving various operations from the doctor D, and the like. The display 36 is, for example, a liquid crystal display, a cathode ray tube (CRT), an organic electroluminescence (EL) display, or the like. The display 36 may be of a desktop type, or may be a display device (for example, a tablet terminal) that can wirelessly communicate with the main body of the terminal device 3.

### [PACS]

Referring back to FIG. 1, the PACS 5 manages various types of medical image data. The PACS 5 stores and manages, for example, clinical data CD obtained by various modalities M, analysis data AD which is analysis results obtained by the analysis server 1, and the like.

### [Report System]

The report system 7 stores and manages report data RD created on the basis of operations by the doctor D via the terminal device 3. FIG. 5 is a diagram showing an example of report data RD stored in the report system 7 according to an embodiment. In the report data RD, for example, an analysis application that is an output source of an analysis result adopted as a key image, order information, modality information, diagnosis information of the doctor D, and the like are associated with a subject ID that identifies a subject. The report data RD may include numerical information of analysis results.

### [HIS]

The HIS 9 is a computer system that provides operational support within a hospital. The HIS 9 has various subsystems. The various subsystems include, for example, an electronic medical record system 91, an order system 93, and the like. The doctor D can refer to an electronic medical record of a subject by using the electronic medical record system 91 via the terminal device 3. Further, the doctor D can order various medical image diagnoses by using the order system 93 via the terminal device 3.

### [Report Data Registration Processing]

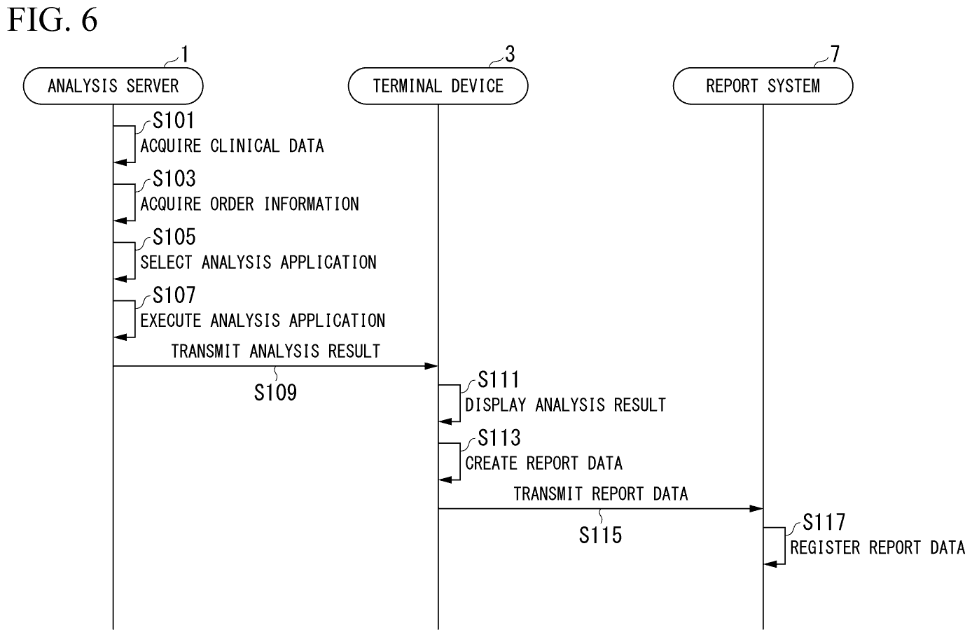

Next, a flow of processing of the medical information processing system S will be described. FIG. 6 is a sequence diagram showing an example of processing of registering report data RD in the medical information processing system S according to the embodiment. The registration processing shown in FIG. 6 is started, for example, when the doctor D inputs an instruction to start the registration processing via the input interface 34 of the terminal device 3.

First, the acquisition function 101 of the analysis server 1 acquires clinical data of a target subject from the modality M (step S101). For example, the analysis server 1 acquires a CT image from the modality M, which is an X-ray CT device. A DICOM tag is attached to this CT image.

Subsequently, the acquisition function 101 of the analysis server 1 acquires order information with respect to the subject from the order system 93. This order information is issued at the time of instructing acquisition of the clinical data (CT images) acquired in step S101. That is, the order information is registered in the order system 93 when the doctor D (or another doctor) orders acquisition of the clinical data (CT image) before this processing of registering the report data RD is started. This order information includes, for example, information such as the purpose of an examination. The acquisition function 101 may acquire information on the electronic medical record of the subject from the electronic medical record system 91. This electronic medical record includes, for example, information on the results of other examinations performed on the subject in the past.

Subsequently, the selection function 103 of the analysis server 1 selects one or more analysis applications to be executed from among a plurality of types of analysis applications stored in advance in the memory 12 on the basis of the clinical data (DICOM tag information), order information, electronic medical record information, and the like acquired by the acquisition function 101 (step S105). The selection function 103 selects one or more analysis applications to be executed from among the plurality of analysis applications using, for example, a learning model MD stored in the memory 12.

Subsequently, the execution function 105 of the analysis server 1 executes the analysis application selected by the selection function 103 (step S107). When a plurality of analysis applications are selected by the selection function 103, the execution function 105 executes each of the plurality of analysis applications and obtains each analysis result.

Subsequently, the provision function 107 of the analysis server 1 transmits the analysis results output from the analysis application executed by the execution function 105 to the terminal device 3 (step S109).

Subsequently, the display control function 307 of the terminal device 3 executes the viewer application VA stored in advance in the memory 32 in response to an operation of the doctor D via the input interface 34, and causes the display 36 to display the analysis results transmitted from the analysis server 1 (step S111). Accordingly, the doctor D can check the analysis results displayed on the display 36.

Subsequently, the determination function 303 and the report function 305 of the terminal device 3 execute the report creation application RA stored in advance in the memory 32 in response to an operation of the doctor D via the input interface 34, and create report data (step S113). The report data includes, for example, an analysis application that is an output source of an analysis result that has been adopted as a key image, an analysis application that is an output source of an analysis results that has not been adopted as a key image, order information, modality information, diagnosis of the doctor D, and the like.

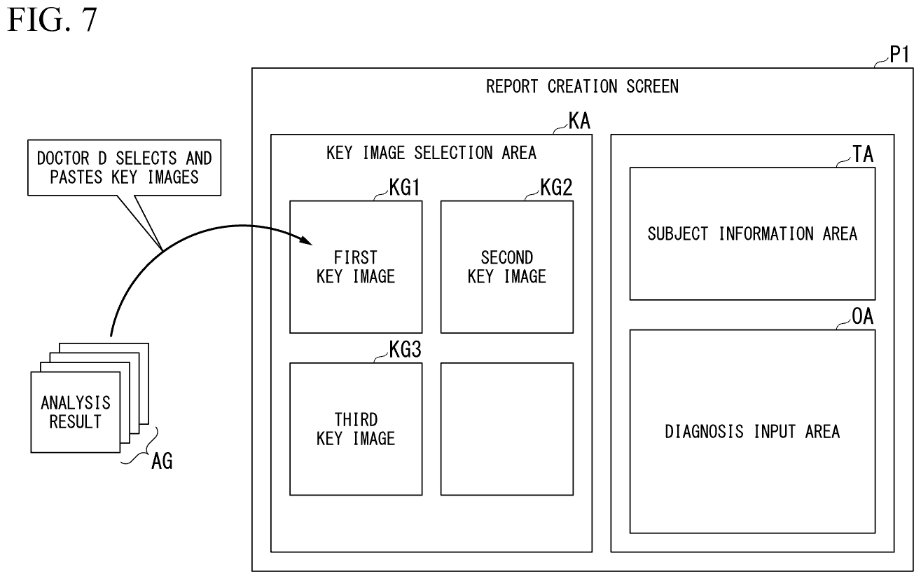

FIG. 7 is a diagram showing an example of a report creation screen P1 displayed on the terminal device 3 according to an embodiment. The report creation screen P1 is provided with, for example, a key image selection area KA, a subject information area TA, and a diagnosis input area OA. The key image selection area KA is an area for receiving an image determined by the doctor D to be useful from among analysis results AG as a key image. The doctor D pastes a key image into the key image selection area KA by operating a mouse, which is the input interface 34, to perform a click-and-drop operation. Alternatively, the doctor D operates the input interface 34 to input hyperlink information of an analysis result determined as a key image into the key image selection area KA. The determination function 303 determines a key image according to such an operation performed by the doctor D. The subject information area TA is an area for displaying various types of information on a subject included in order information and electronic medical record information. The diagnosis input area OA is an area for receiving input of diagnosis of the doctor D in response to an operation of the doctor D via the input interface 34. Further, information on an evaluation level may be added to the determined key image in accordance with an operation of the doctor D. The evaluation level may be represented as a high or low level such as “very good,” “good,” or “average,” or may be represented as a numerical value (for example, a value from 1 to 5).

The report creation screen P1 may be provided with an area (non-key image selection area) into which an analysis result (non-key image) that has not been selected as a key image is pasted. A non-key image is an image determined by the doctor D to be an invalid analysis result (an analysis result output by an inappropriate analysis application). The doctor D pastes a non-key image into the non-key image selection area by operating the mouse, which is the input interface 34, to perform a click-and-drop operation. Alternatively, the doctor D operates the input interface 34 to input hyperlink information of an analysis result determined as a non-key image into the non-key image selection area. The determination function 303 determines a non-key image in response to such an operation performed by the doctor D.

Subsequently, the report function 305 of the terminal device 3 transmits the created report data to the report system 7 (step S115). The report system 7 performs processing of registering the report data received from the terminal device 3 (step S117). Accordingly, the report data registration processing ends.

### [Report Data Learning Processing]

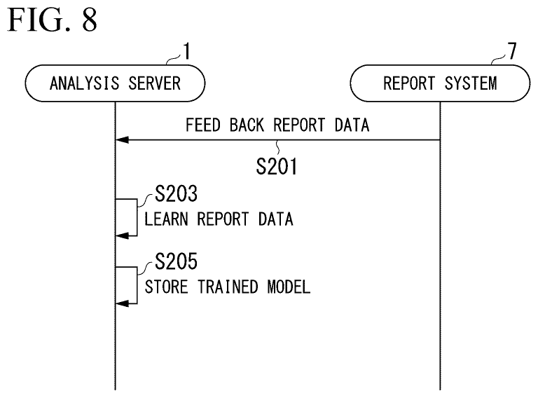

FIG. 8 is a sequence diagram showing an example of learning processing in the medical information processing system S according to the embodiment. The learning processing shown in FIG. 8 is started, for example, when an administrator of the analysis server 1 inputs an instruction to start the learning processing via the input interface (not shown) of the analysis server 1, or according to batch processing at a predetermined date and time.

First, the report system 7 performs feedback by transmitting registered report data (feedback information) to the analysis server 1 (step S201). The report data transmitted to the analysis server 1 includes, for example, an analysis application that is an output source of an analysis result adopted as a key image, an analysis application that is an output source of an analysis result that has not been adopted as a key image, order information, modality information, diagnosis, and the like.

Subsequently, the learning function 109 of the analysis server 1 performs learning processing using the report data received from the report system 7 to generate a learning model MD (step S203). The learning function 109 stores the generated trained learning model MD in the memory 12 (step S205). Accordingly, the report data learning processing ends.

If the report data includes information on an analysis application that is an output source of an analysis result that has not been adopted as a key image, the learning function 109 performs learning processing such that a degree of contribution (weight) of the analysis application to output data of the model decreases and generates the learning model MD. Alternatively, even if the report data does not include information on an analysis application that is an output source of an analysis results that has not been adopted as a key image, the learning function 109 may identify the analysis application that is an output source of the analysis result that has not been adopted as a key image from a list information of analysis results provided to the doctor D and information on analysis results adopted by the doctor D as key images, perform learning processing such that a degree of contribution (weight) of the identified analysis application to the output data of the model decreases, and generate the learning model MD.

The report data may include information on a reference time or the number of references of each analysis result in the terminal device 3 by the doctor D, and the report system 7 may feed this information back to the analysis server 1. In this case, the selection function 103 of the analysis server 1 may select one or more analysis applications on the basis of the information on the reference time or the number of references of each analysis result. As a result, an analysis result that has not been selected as a key image by the doctor D but has a long reference time or a large number of references may be determined to be likely to be useful and may be considered in subsequent selection.

The number of learning models MD stored in the memory 12 is not limited to one. A plurality of types of learning models MD having different numbers of pieces and types of input/output data may be stored in the memory 12 as shown in FIG. 3A to FIG. 3C. The selection function 103 of the analysis server 1 may select an analysis application using one learning model or a plurality of learning models selected by a user from among a plurality of types of learning models MD.

In addition, at the time of selecting an analysis application using a trained model, the selection function 103 of the analysis server 1 may evaluate whether an analysis application close to data at the time of learning can be proposed, thereby calculating the reliability of proposal. If the reliability is lower than a predetermined threshold value, the trained model caused to propose an analysis application may be switched to a trained model that uses the minimum number of input items and proposal may be performed again upon determining that there are excessively many input data items to be evaluated (a situation in which similar input data has not been learned or a situation in which training data is insufficient) and a proposal for a stable analysis application cannot be obtained.

That is, learning models include a plurality of learning models having different numbers of pieces or types of input data, and the selection function 103 may select one or more learning models from a plurality of learning models according to acquired clinical data and select one or more analysis applications.

According to the embodiment described above, it is possible to appropriately and easily select an analysis application for clinical data by acquiring clinical data of a subject and selecting one or more analysis applications that analyze the acquired clinical data on the basis of report data of a user regarding analysis results of a plurality of types of analysis applications that analyze the clinical data.

### SUPPLEMENTARY NOTE

As another embodiment, the medical information processing device of the embodiment can also be represented as a program that causes a computer to acquire clinical data of a subject and to select one or more analysis applications that analyze the acquired clinical data on the basis of report data of a user regarding analysis results of a plurality of types of analysis applications that analyze the clinical data.

While certain embodiments have been described, these embodiments have been presented by way of example only, and are not intended to limit the scope of the inventions. Indeed, the novel embodiments described herein may be embodied in a variety of other forms; furthermore, various omissions, substitutions, and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing device comprising processing circuitry configured to:
acquire clinical data of a subject; and
select one or more analysis applications that analyze the acquired clinical data on the basis of report data of a user regarding analysis results of a plurality of types of analysis applications that analyze clinical data.

2. The medical information processing device according to claim 1, wherein the processing circuitry is configured to select the one or more analysis applications on the basis of types of analysis applications that have generated the analysis results selected by the user in the report data.

3. The medical information processing device according to claim 2, wherein the processing circuitry is configured to select the one or more analysis applications on the basis of supplementary information associated with the analysis results included in the report data in addition to the types of the analysis applications.

4. The medical information processing device according to claim 3, wherein the supplementary information includes at least one of information indicating diagnosis of the user or order information of the clinical data.

5. The medical information processing device according to claim 1, wherein the processing circuitry is configured to input the acquired clinical data to a learning model trained to output candidates for analysis applications that analyze the clinical data when the clinical data is input to select the candidates for the analysis applications output from the learning model as the one or more analysis applications.

6. The medical information processing device according to claim 5, wherein the processing circuitry is further configured to generate the learning model by learning the report data.

7. The medical information processing device according to claim 1, wherein the processing circuitry is configured to select the one or more analysis applications on the basis of statistical data of the analysis applications that have generated the analysis results included in the report data.

8. The medical information processing device according to claim 1, wherein the processing circuitry is further configured to execute the selected one or more analysis applications and provide analysis results of the executed one or more analysis applications.

9. The medical information processing device according to claim 1, wherein the processing circuitry is configured to acquire the report data from a report system.

10. The medical information processing device according to claim 1, wherein the processing circuitry is configured to select the one or more analysis applications on the basis of a reference time or a number of references of the analysis results by the user.

11. The medical information processing device according to claim 5, wherein the learning model includes a plurality of learning models having different numbers of pieces or types of input data, and
wherein the processing circuitry is configured to select one or more learning models from the plurality of learning models according to the acquired clinical data, and select the one or more analysis applications.

12. A medical information processing system comprising a medical information processing device and a report system,
wherein the report system is configured to store report data created by a user in the past regarding analysis results of a plurality of types of analysis applications that analyze clinical data, and
the medical information processing device comprises processing circuitry configured to:
generate a learning model that outputs candidates for analysis applications that analyze clinical data when the clinical data is input by learning the report data stored in the report system; and
acquire new clinical data of a subject to be analyzed and input the acquired new clinical data of the subject to be analyzed to the generated learning model to select the candidates for the analysis applications output from the learning model as one or more analysis applications that analyze the acquired new clinical data of the subject to be analyzed.

13. A medical information processing system comprising a medical information processing device and a report system,
wherein the report system is configured to store report data created by a user in the past regarding analysis results of a plurality of types of analysis applications that analyze clinical data, and
the medical information processing device comprises processing circuitry configured to:
acquire new clinical data of a subject to be analyzed; and
select one or more analysis applications that analyze the acquired new clinical data of the subject to be analyzed on the basis of statistical data of analysis applications that have generated the analysis results included in the report data.

14. A medical information processing method, using a computer, comprising:
acquiring clinical data of a subject; and
selecting one or more analysis applications that analyze the acquired clinical data on the basis of report data of a user regarding analysis results of a plurality of types of analysis applications that analyze clinical data.
